(19) United States
(12) Patent Application Publication
(10) Pub. No.: US 2014/0132918 A1
(43) Pub. Date: May 15, 2014

(54) OPHTHALMOLOGIC APPARATUS AND METHOD

(71) Applicant: CANON KABUSHIKI KAISHA, Tokyo (JP)

(72) Inventor: Satoshi Aikawa, Yokohama-shi (JP)

(73) Assignee: CANON KABUSHIKI KAISHA, Tokyo (JP)

(21) Appl. No.: 14/066,900

(22) Filed: Oct. 30, 2013

(30) Foreign Application Priority Data
Nov. 9, 2012 (JP) 2012-247029

(51) Int. Cl. A61B 3/12

(52) U.S. Cl. CPC A61B 3/12
USPC 351/206; 351/246

(57) ABSTRACT

An ophthalmologic apparatus obtains a suitable defocus state early in split AF even immediately after the apparatus shifts to an fundus observation state. At the time of focus state detection immediately after the shift to the fundus observation state, the apparatus tarns the fundus observing illumination light off if a focus determination unit determines that the defocus amount is equal to or more than a predetermined value, and turns the fundus observing illumination light on if the focus determination unit determines that an in-focus state immediately before photographing is obtained, thereby allowing the examiner to check a fundus state immediately before photographing.

### BACKGROUND OF THE INVENTION

1. Field of the Invention

The present invention relates to an ophthalmologic apparatus and an ophthalmologic method.

2. Description of the Related Art

With regard to ophthalmologic apparatuses, in order to improve the accuracy of automatic focusing or automatic alignment, Japanese Patent Application Laid-Open No. H05-95907 has proposed a technique of, when performing detection processing using index light beams, improving the delectability of the index light beams by dimming other light beams.

When executing split AF (split automatic focusing operation to be referred to as split AF or AF hereinafter) based on the evaluation value of split indices, if the defocus amount is large before the execution of AF, a fundus observation image sometimes blurs considerably to become a visually undesirable image. In this case, a split index image may have a large blur, and the accuracy of the calculated evaluation value of the split indices may deteriorate.

### SUMMARY OF TEE INVENTION

The present invention has been made in consideration of this situation, and provides an ophthalmologic apparatus and method which can stably obtain a suitable in-focus state when executing split AF.

According to one aspect of the present invention, there is provided an ophthalmologic apparatus which performs split automatic focusing by using at least one pair of focus indices projected on a fundus of an eye to be inspected and picks up a fundus image of the fundus by using an image pickup unit, the apparatus comprising a fundus illumination unit configured to illuminate the fundus, a focusing unit configured to perform the split automatic focusing, a focus state determination unit configured to determine a focus state obtained by the focusing unit, and a control unit configured to control illumination for the fundus provided by the fundus illumination unit in accordance with a determination result obtained by the focus state determination unit.

According to another aspect of the present invention, there is provided an ophthalmologic method of performing split, automatic focusing by using at least one pair of focus indices projected on a fundus of an eye to be inspected and picking up a fundus image of the fundus by using an image pickup unit upon performing focusing, the method comprising a focusing step of causing a focusing unit to perform the split automatic focusing, a focus state determination step of determining a focus state obtained by the focusing unit, and a control step of controlling illumination for the fundus provided by a fundus illumination unit in accordance with a determination result obtained in the focus state determination step.

According to the present invention, first, when executing first AF for a fundus image, the operator can observe the image without recognizing any visually undesirable fundus image with a large defocus amount or feeling discomfort by turning fundus observation light intensity off or dimming. Second, when executing first AF, it is possible to more accurately evaluate blurred split indices by turning fundus observation light intensity off or dimming.

Further features of the present invention will become apparent from the following description of exemplary embodiments with reference to the attached drawings.

### BRIEF DESCRIPTION OF THE DRAWINGS

FIG. 1 is a schematic view for explaining an ophthalmologic apparatus according to an embodiment of the present invention.

FIG. 2 is an electrical block diagram for explaining the ophthalmologic apparatus according to the embodiment of the present invention.

FIG. 3 is a flowchart for explaining an ophthalmologic method according to the embodiment of the present invention.

FIG. 4 is a view showing the relationship between the defocus amount and the split image.

FIG. 5 is a flowchart showing an overall photographing sequence.

### DESCRIPTION OF THE EMBODIMENTS

Preferred embodiments of the present invention will now be described in detail in accordance with the accompanying drawings.

### First Embodiment

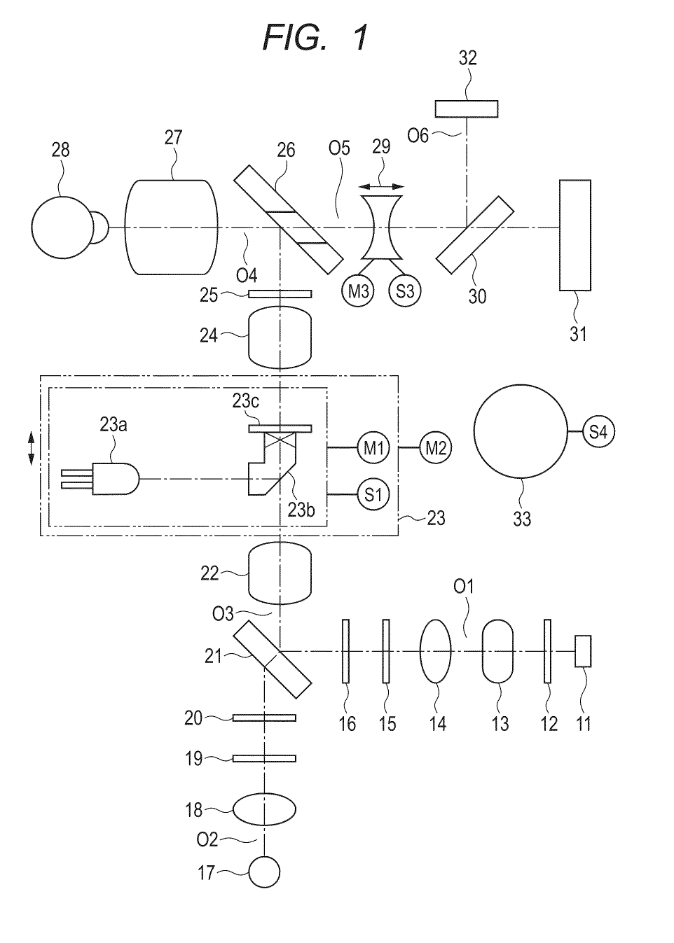

A fundus camera as an ophthalmologic apparatus according to the first embodiment of the present invention and a photographing method using it will be described in detail with reference to FIGS. 1 to 4.

FIG. 1 is a schematic view for explaining the first embodiment of the present invention.

This fundus camera is roughly constituted by a photographing light source portion O1, an observation light source portion O2, an illumination optical system O3, a photographing/illumination optical system O4, a photographing optical system O5, and an internal fixation target portion O6. The light beam emitted by the photographing light source portion O1 or the observation light source portion O2 illuminates the fundus portion of an object through the illumination optical system O3 and the photographing/illumination optical system O4. The corresponding image is formed on the image pickup element through the photographing/illumination optical system O4 and the photographing optical system O5.

The photographing light source portion O1 generates ring illumination of white light by the following arrangement. A light intensity detection unit 11 is a sensor using a known photoelectric conversion element such as an SPC or PD. A mirror 12 is formed from a glass plate deposited with aluminum or silver, an aluminum plate, or the like. A photographing light source 13 emits light upon application of a voltage to Xe sealed in a glass tube and can obtain white light with sufficient intensity to record a fundus image at the time of photographing. Recently, an increase in the light intensity of an LED has been promoted, and even an annularly arranged LED array can implement the photographic light source 13.

A photographing condenser lens 14 is a general spherical lens. A photographing ring slit 15 is a flat panel having an annular opening. A photographing crystalline lens baffle 16 is also a flat panel having an annular opening. The light beams emitted from the Xe tube 13 include a light beam traveling toward the fundus and a light beam emitted to the opposite side and reflected by the mirror 12 to travel toward the fundus. Therefore, the emitted light intensity of the photographing light source 13 can be lower than that of a light source without the mirror 12. The mirror 12 is formed planar to prevent any light unevenness and impose no distance restriction on the photographing light source 13. The light beam is further condensed toward the fundus by the photographing condenser lens 14. The photographing ring slit 15 then shapes the light into an annular shape when it passes through the anterior ocular segment. In addition, the photographing crystalline lens baffle 16 limits the light beam projected onto the crystalline lens of the eye to be inspected to prevent the generation of unnecessary reflected light from the crystalline lens of the eye on a fundus image

The observation light source portion O2 generates ring illumination of infrared light by the following arrangement. An observation light source 17 is a light source such as a halogen lamp or LED capable of continuously emitting light and emits infrared light based on element characteristics or through an optical filter. An observation condenser lens 18 is a general spherical lens. An observation ring slit 19 is a flat panel having an annular opening. An observation crystalline lens baffle 20 is also a flat panel having an annular opening. The observation light source portion O2 is different from the photographing light source portion O1 only in the type of the light source. The observation condenser lens 18 condenses a light beam. The observation ring slit 19 shapes the light beam at the anterior ocular segment. The observation crystalline lens baffle 20 prevents the generation of reflected light from the crystalline lens to a fundus image.

The illumination optical system O3 relays the light beam generated by the photographing light source portion O1 and the observation light source portion O2 and generates an index image for focusing a fundus image. A dichroic mirror 21 transmits infrared light and reflects visible light. The dichroic mirror 21 reflects the light beam of visible light generated by the photographing light source portion O1 and transmits the light beam of infrared light generated by the observation light source portion O2, thereby guiding the light beams to the illumination optical system O3. An illumination relay lens 1 22 and an illumination relay lens 2 24 form ring illumination into an image on the eye to be inspected. A split unit 23 includes a focus index light source 23a for projecting focus indices, a prism 23b for splitting a light source, and a focus index mask 23c indicating the outer shapes of focus indices. The split unit 23 further includes a drive mechanism for shifting/driving focus indices in the optical axis direction by entering the observation light source portion O2 and moving in the arrow direction in FIG. 1 at the time of observation and an entering/retracting mechanism for retraction from the illumination optical system O3 at the time of photographing. These components for illuminating the fundus constitute a fundus illumination unit for illuminating the fundus in the present invention.

A split shift drive motor M1 shifts/drives the split unit 23 to focus the focus indices. A split position sensor S1 detects the stop position of the split unit 23. A split back and forth drive motor M2 causes the split unit 23 to enter/retract with respect to the illumination optical system O3. The split back and forth drive motor M2 causes the split unit 23 to enter the illumination optical system O3 to project spilt indices in an observation image at the time of fundus observation and causes the split unit 23 to retract from the illumination optical system O3 at the time of photographing, thereby performing control to prevent the focus indices from being reflected in a photographed image. A cornea baffle 25 prevents the generation of unnecessary reflected light from the cornea of the eye to be inspected to a fundus image.

The photographing/illumination optical system O4 projects an illumination light beam on the fundus of an eye 28 to be inspected and outputs a fundus image of the eye. A perforated mirror 26 has its outer circumference formed as a mirror and a hole formed in its center. The light beam guided from the illumination optical system O3 is reflected by the mirror portion and illuminates the fundus of the eye through an objective lens 27. The illuminated fundus image of the eye returns to the objective lens 27 and is output to the photographing optical system O5 through the central hole of the perforated mirror 26.

The photographing optical system O5 forms a fundus image of the eye on an image pickup element upon focal point adjustment of the image. A focus lens 29 adjusts the focal point of a photographing light beam passing through the central hole of the perforated mirror 26. The focus lens 29 moves in the arrow directions in FIG. 1 to perform focal point adjustment. A focus lens drive motor M3 drives the focus lens 29 to perform focusing. A focus lens position sensor S3 detects the stop position of the focus lens 29. An image pickup element 31 photoelectrically converts photographing light. A processing circuit (not shown) A/D-converts the electrical signal obtained by the image pickup element 31 of the image pickup unit into digital data, which is displayed on a display device (not shown) at the time of infrared observation and recorded on a recording medium (not shown).

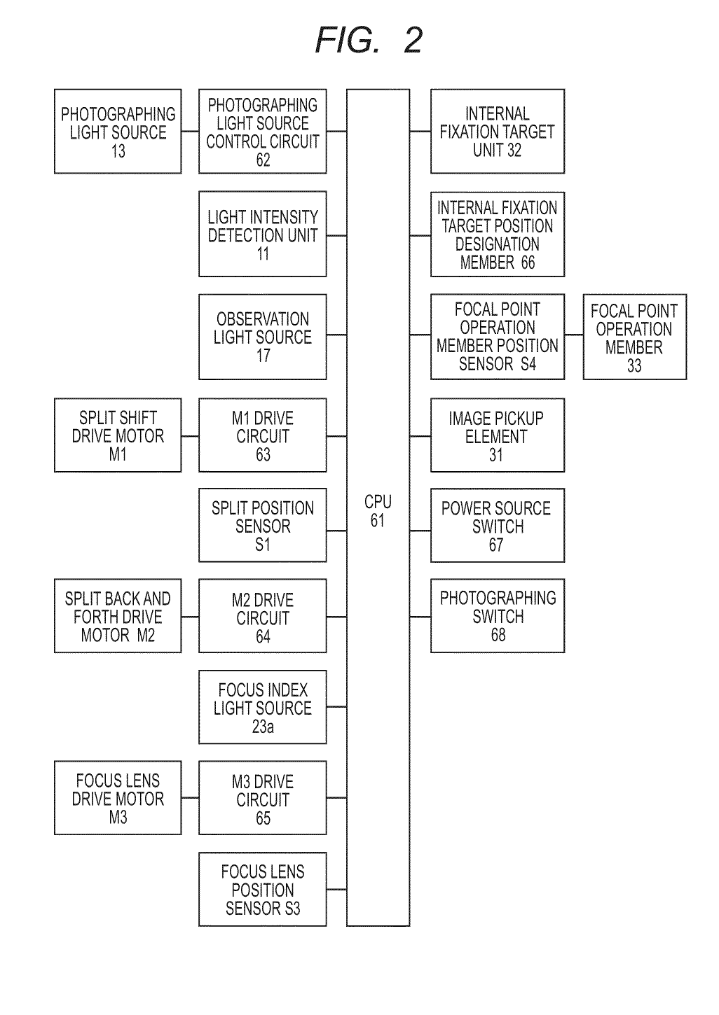

The internal fixation target portion O6 includes an internal fixation target unit 32 facing an optical path split from the photographing optical system O5 by a half mirror 30. The internal fixation target unit 32 is constituted by a plurality of LEDs and turns on an LED at a position corresponding to a visual fixation portion selected by an examiner using an internal fixation target position designation member 66 (see FIG. 2). When an object fixes its view on the turned-on LED, the examiner can acquire a fundus image in a desirable direction.

When the examiner operates a focal point operation member 33, it is possible to detect the stop position of the focal point operation member 33 by using a focal point operation member position sensor S4.

FIG. 2 is an electrical block diagram, concerning the photographing unit of the fundus camera which exemplifies the first embodiment of the present invention. A CPU 61 controls all the operation of the fundus camera. A photographing light source control circuit charges the photographing light source 13 with energy for light emission before photographing and discharges the charged electric energy to make the photographing light source 13 emit light at the time of photographing. An M1 drive circuit 63 drives the split shift drive motor M1 to move the split unit 23 to a position corresponding to an output from the focal point operation member position sensor S4.

An M2 drive circuit drives the split back and forth drive motor M2 to make the split unit 23 enter/retract with respect to the observation light source portion O2 before and after photographing. Like an M2 drive circuit 64, an M3 drive circuit drives the focus lens drive motor M3 to move the focus lens 29 to a position corresponding to an output from the focal point operation member position sensor S4. A power source switch 67 is a switch for selecting a power state of the fundus camera. A photographing switch 68 is a switch for executing photographing by using the fundus camera.

The overall fundus camera according to the first embodiment of the present invention has been described so far. The AF operation of the fundus camera according to the first embodiment of the present invention will be described in detail below.

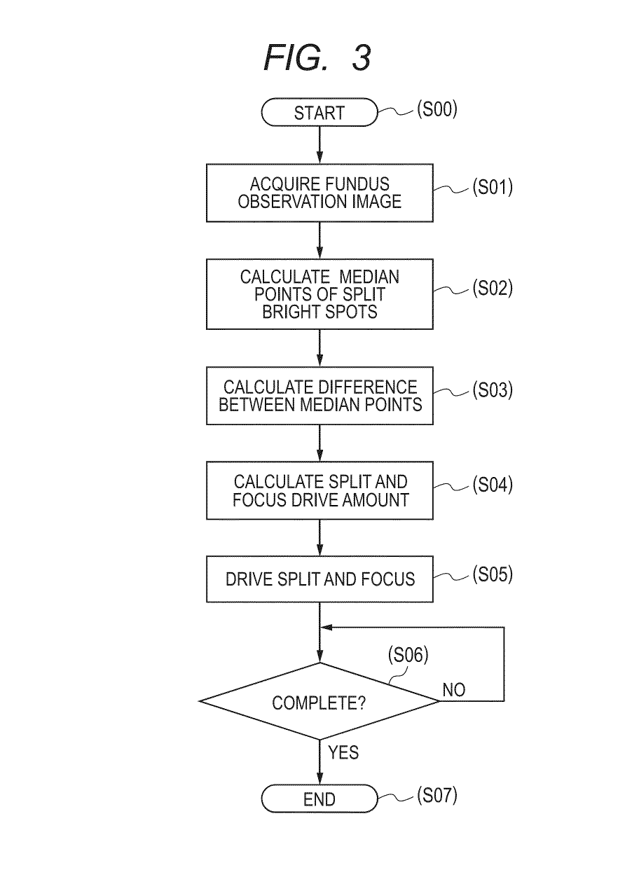

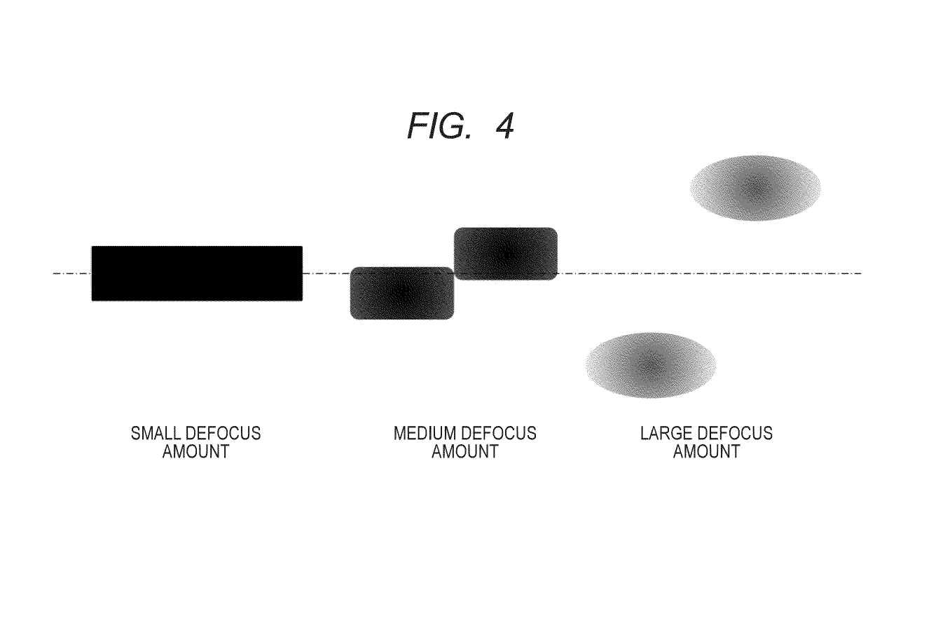

FIGS. 3 and 4 are respectively a flowchart concerning AF operation in the first embodiment of the present invention and a view showing the relationship between the defocus amount and the split image.

A procedure for AF operation will be described based on FIG. 3.

In step S00, the CPU 61 starts the procedure.

In step S01, the CPU 61 acquires the fundus observation image obtained from the image pickup element 31.

In step S02, the CPU 61 extracts two split bright spots from the acquired observation image and calculates the median points of the respective split bright spots.

In step S03, the CPU 61 obtains the difference in the y-axis direction between the median points of the two split bright spots.

In step S04, the CPU 61 calculates the split and focus drive amount to achieve an in-focus state with respect to the current split and focus state from the difference between the two bright spots.

In step S05, the CPU 61 drives the split and focus in the direction obtained in step S04. Upon determining in step S06 that drive operation corresponding to the drive amount obtained in step S04 is complete, the CPU 61 terminates the procedure in step S07.

Both the focus and split are at rest at the time of step S07. The above operation is executed by a focusing unit constituted by the respective components described above for performing split automatic focusing in the present invention, for example, an M3 drive circuit 65 and the focus lens position sensor S3.

The relationship between the defocus amount and the split image will be described with reference to FIG. 4.

The defocus amounts sequentially increase from the left to the right. The left view indicates a state with a small defocus amount, in which the left and right split bright spots are aligned without any shift in the y direction. The split bright spots are in an in-focus state and hence displayed sharply. The central view indicates a state with a defocus amount slightly larger than that in the left view, in which the left and right split bright spots are vertically shifted from each other. Likewise, with an increase in defocus amount, the split bright spots are displayed less sharply, with its peripheral portion blurring. The right view indicates a state with a very large defocus amount, in which the vertical shift amount of the left and right split bright spots is very large. At the same time, each split bright spot considerably blurs.

If the defocus amount increases in this manner, the split bright spots blur and submerge in the background image. When, therefore, extracting split bright spots and calculating the median points of them in step S02 in FIG. 3, it is difficult to extract only split bright spot information by erasing background information from the image. As a result, the reliability of the calculated defocus amount deteriorates.

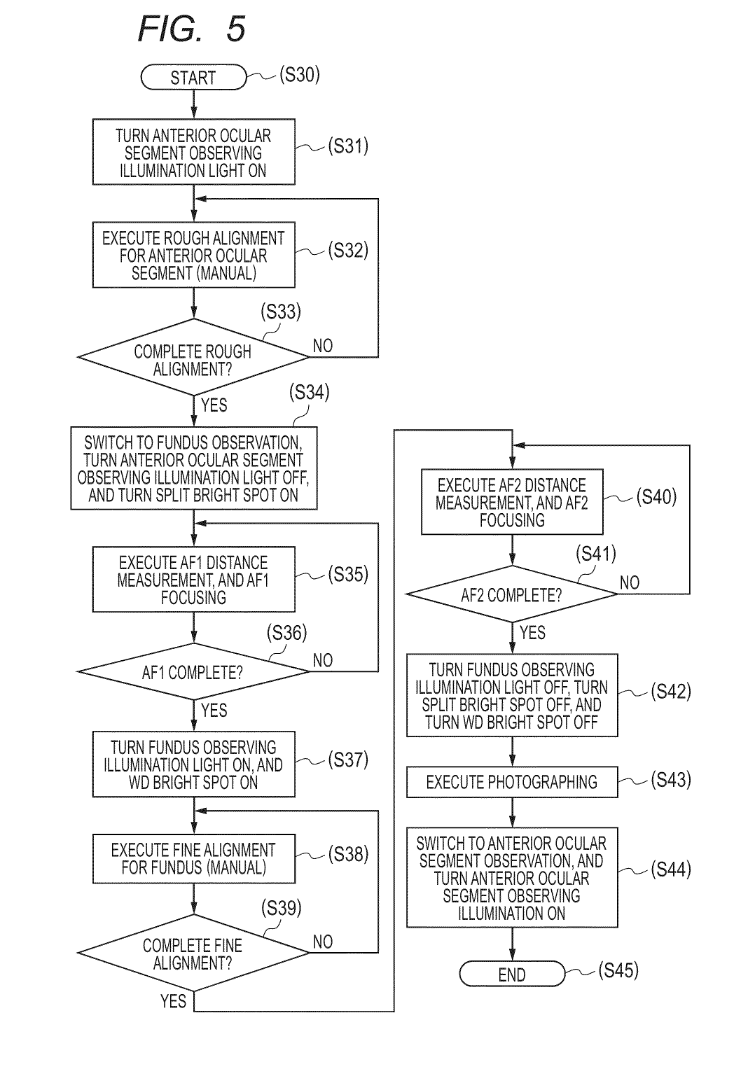

FIG. 5 is a flowchart showing an overall photographing sequence.

The overall photographing sequence will be described based on FIG. 5.

In step S30, the CPU 61 starts the photographing sequence.

In step S31, first of all, the CPU 61 turns the anterior ocular segment observing illumination light on. This allows the examiner to observe the anterior ocular segment of the object.

In step S32, the examiner performs anterior ocular segment alignment. The examiner makes adjustment to locate the pupil in the center of the screen while adjusting the operating distance so as to match anterior ocular segment splits (not shown).

In step S33, the fundus camera determines the state of the anterior ocular segment alignment. If the anterior ocular segment alignment is not complete, the process returns to step S32. If the anterior ocular segment alignment is complete, the process advances to step S34.

In step S34, the CPU 61 performs fundus observation switching. More specifically, the CPU 61 retracts the anterior ocular segment observation lens barrel (not shown) to set a fundus observation state. In addition, the CPU 61 turns the anterior ocular segment observing illumination light off and turns the split bright spots on. With this operation, the rough alignment is complete and the split bright spots are projected on the fundus of the object. However, since the CPU 61 does not turn the fundus observing illumination light on, the examiner cannot observe any fundus image.

In step S35, the CPU 61 performs AF1 distance measurement. This operation is for rough focusing on the object fundus image after the rough alignment. Upon completing distance measurement, the CPU 61 drives the split and focus to shift to an in-focus state.

In step S36, the CPU 61 determines whether AF1 is complete. If the difference in the y direction between median points of the two split bright spots is equal to or less than a predetermined value, the CPU 61 determines that AF1 is complete, and the process advance to step S37. If the difference is larger than the predetermined value, the process returns to step S35 to perform AF1 operation again. The above operation is executed by a module area, in the CPU 61, which functions as a focus state determination unit for determining a focus state. In addition, the predetermined value for the difference in y direction described above corresponds to the first threshold value in the present invention.

In step S37, the CPU 61 turns the fundus observing illumination light on and also turns the WD bright spot on. That is, if the determination result obtained by the focusing determination unit is equal to or less than the first threshold value, the CPU 61 as a control unit executes control to turn the fundus illumination unit and the fundus observing illumination light on. In this state, the examiner can observe both the fundus image and the WD bright spot, and hence can perform fine alignment in accordance with the position/defocus amount of the WD bright spot.

In step S38, the examiner manually performs fundus fine alignment.

In step S39, the CPU 61 determines whether the fundus fine alignment is complete. The CPU 61 extracts a WD image from the fundus observation image obtained from the image pickup element 31, evaluates the position/defocus amount of the image in the fundus camera, recognizes a fundus fine alignment state, and determines whether the processing is complete. If the processing is complete, the process advances to step 340. If the processing is not complete, the process returns to step S38.

In step S40, the CPU 61 performs AF2 distance measurement. This operation is distance measurement for the execution of final AF immediately before photographing. The CPU 61 further drives the split and focus to shift to an in-focus state.

In step S41, the CPU 61 determines whether AF2 is complete. This operation is final AF based on a distance measurement result in a state with a small defocus amount after the completion of AF1. For this reason, this determination criterion is preferably severer than the determination criterion in step S36. This determination criterion corresponds to the second threshold value in the present invention. If the determination result on a focus state is equal to or less than the second threshold value, the process shifts to the next step. Therefore, the first threshold value is set to be larger than the second threshold value. For the same reason, it is possible to shorten the distance measurement time by narrowing the split determination range in AF2 distance measurement (step S41) compared with that in AF1 distance measurement (step S35). That is, the first focus state detection area as a detection area for a focus state when comparing the first threshold value with a determination result is preferably set narrower than the second focus state detection area as a detection area for a focus state when comparing the second threshold value with a determination result.

In step S42, the CPU 61 prepares for photographing by turning the fundus observing illumination light, split bright spots, and WD bright spot off.

In step S43, the CPU 61 performs final photographing operation.

In step S44, the CPU 61 inserts the anterior ocular segment observation lens barrel (not shown), turns the anterior ocular segment observing illumination light on, and shifts the fundus camera to the anterior ocular segment observation state in order to prepare for the next photographing operation. The process then advances to step S45 to complete the sequence.

In this sequence, the CPU 61 turns only the split bright spots on but turns the fundus observing illumination light and the WD bright spot off in AP1 in step S35. In contrast, in AF2 in step S40, the CPU 61 turns the fundus observing illumination light and the WD bright spot on as well as the split bright spots. This is because the de focus amount may be large at the time of AF1, and an image displayed for the examiner may considerably blur and hence worthless to be seen, even if it is displayed. In addition, as shown in FIG. 4, if the defocus amount is large, since the split bright spots considerably blur, turning the fundus observing illumination light on and depicting a fundus image may degrade the reliability in evaluating the split bright spots. In AF2, however, the examiner can confirm that the fundus camera performs photographing while the image having undergone alignment immediately before photographing is finally in better focus.

In addition, when performing AF2, since AF1 is complete, the defocus amount is small, and it is possible to accurately grasp the split bright spots even with a fundus image in the background. Providing different illumination states in AF1 and AF1 based on such difference in state can implement user-friendly, accurate AF.

When, for example, executing split AF (automatic focusing operation to be referred to as AF hereinafter) based on the evaluation value of split indices, dimming other light beams indicates that it is not possible to perform other preparing operations such as alignment and fixation guidance at the time of AF operation. That is, this produces a state in which the operator cannot perform operation other than focusing against his/her will, resulting in poor usability. The present invention can also make improvements concerning such poor usability.

In addition, when performing second and subsequent AF, providing illumination with a fundus observation light intensity optimal for fundus observation enables to always perform operations other than focusing. Furthermore, it is possible to present an optimal image allowing the operator to grasp an AF state.

### Other Embodiments

The first embodiment has exemplified the case in which the fundus camera turns the fundus observing illumination light off in the first distance measurement until an in-focus state is achieved.

However, it is not always necessary to turn the illumination light off until an in-focus is achieved. It is possible to achieve the object to improve the distance measurement accuracy if the illumination light is turned off at least at the time of distance measurement. With regard to the object to inhibit a considerably blurred image from being displayed, it is possible to express focusing operation by the following several operations with some design effects, for example: (1) turning the illumination light on during focusing operation when the defocus amount becomes a predetermined value or less; (2) turning the illumination light on when a predetermined period of time has elapsed since the start of focusing operation; and (3) gradually increasing the luminance of the fundus observing illumination light after the completion of distance measurement.

The present invention is also implemented by executing the following processing. That is, this is the processing of supplying software (programs) for implementing the functions of the above embodiments to a system or apparatus via a network or various types of storage media and making the computer (or the CPU, MPU, or the like) of the system or apparatus read out and execute the software.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-247029, filed Nov. 9, 2012,which is hereby incorporated by reference herein in its entirety.

What is claimed is:

1. An ophthalmologic apparatus which performs automatic focusing by using a focus index projected on a fundus of an eye to be inspected and picks up a fundus image of the fundus by using an image pickup unit, the apparatus comprising:
a fundus illumination unit configured to illuminate the fundus;
a focusing unit configured to perform the automatic focusing;
a focus state determination unit configured to determine a focus state obtained by the focusing unit; and
a control unit configured to control illumination for the fundus provided by the fundus illumination unit in accordance with a determination result obtained by the focus state determination unit.

2. An apparatus according to claim 1, wherein the control unit sets an observation state of the fundus by turning the fundus illumination unit on if the determination result obtained by the focus determination unit is not more than a first threshold value, and
the control unit sets a photographing state of the fundus by turning the fundus illumination unit off if the determination result is not more than a second threshold value.

3. An apparatus according to claim 2, wherein the first threshold value is set to be larger than the second threshold value.

4. An apparatus according to claim 2, wherein a second focus state detection area as a focus state detection area where the second threshold value is compared with the determination result is set to be narrower than a first focus state detection area as a focus state detection area where the first threshold value is compared with the determination result.

5. An apparatus according to claim 1, wherein when a defocus amount becomes not more than a predetermined value during operation by the focusing unit, the control unit turns the fundus illumination unit on.

6. An apparatus according to claim 1, wherein, when a predetermined period of time has elapsed since the start of operation by the focusing unit, the control unit turns the fundus illumination unit on.

7. An apparatus according to claim 1, wherein after the focus state determination unit completes operation to obtain an in-focus state, the control unit gradually increases luminance of the fundus illumination unit.

8. An apparatus according to claim 1, wherein the focus index includes at least one pair of focus indices, and the automatic focusing includes split automatic focusing using the pair of focus indices.

9. An ophthalmologic method of performing automatic focusing by using an index projected on a fundus of an eye to be inspected and picking up a fundus image of the fundus by using an image pickup unit, the method comprising:
a focusing step of causing a focusing unit to perform the focusing operation;
a focus state determination step of determining a focus state obtained by the focusing unit; and
a control step of controlling illumination for the fundus provided by the fundus illumination unit in accordance with a determination result obtained in the focus state determination step.

10. A method according to claim 9, wherein an observation state of the fundus is set by turning the fundus illumination unit on if the determination result obtained in the focus determination step is not more than a first threshold value, and
a photographing state of the fundus is set by turning the fundus illumination unit off if the determination result is not more than a second threshold value.

11. A program for causing a computer to execute each step in an ophthalmologic method defined in claim 9.
